(19) United States
(12) Patent Application Publication
(10) Pub. No.: US 2017/0191013 A1
(43) Pub. Date: Jul. 6, 2017

(54) CONTAINER FOR CULTURING ORGANISMS, METHOD FOR MONITORING THE CULTURING OF ORGANISMS INSIDE SAID CONTAINER, AND MONITORING SYSTEM

(71) Applicant: C.C.M. Beheer B.V., Nuenen (NL)

(72) Inventors: Franciscus Henricus Alphonsus Gerardus Fey, Nuenen (NL); Gerard Rudolf Riemens, Nuenen (NL); Edwin Langerak, Nuenen (NL); Marinus Beije, Nuenen (NL)

(21) Appl. No.: 15/313,125

(22) PCT Filed: May 28, 2015
(86) PCT No.: PCT/NL2015/050384

§ 371 Date: Nov. 22, 2016

(30) Foreign Application Priority Data
May 30, 2014 (NL) 2012922

(51) Int. Cl. C12M 1/22; C12M 1/00; G01N 33/487; C12M 1/36; C12M 1/34; C12M 1/32; C12M 3/00

(52) U.S. Cl. CPC C12M 23/10; C12M 23/12; C12M 23/22; C12M 23/38; C12M 23/48; C12M 41/48; C12M 41/46; G01N 33/487

(57) ABSTRACT

A shallow container for culturing organisms includes a holder defining a cavity and a separate lid to close off the cavity. The lid includes a transparent window portion to be arranged above the cavity when the lid closes off the cavity. The window portion includes an upper surface and a lower surface extending parallel to each other; where the cavity is filled with a matrix having a reflective top surface on which an organism to be cultured can be received, and where in at least one position of the lid with respect to the holder, in which the lid closes off the cavity, the top surface of the matrix extends non-parallel to the upper and lower surfaces of the window portion.

### CROSS-REFERENCE TO RELATED APPLICATIONS

This application is the National Stage of International Application No. PCT/NL2015/050384 filed May 28, 2015, which claims the benefit of Netherlands Application No. NL 2012922, filed May 30, 2014, the contents of which is incorporated by reference herein.

### FIELD OF THE INVENTION

The invention relates to a container for culturing organisms, a method for monitoring the culturing of organisms inside said container and a monitoring system.

### BACKGROUND OF THE INVENTION

Examples of such containers are petri-dishes and microtiter plates usually comprising a holder and a separate lid, said holder defining a cavity for receiving organisms and said lid being configured to close off the cavity.

When culturing microbes, the cavity of the holder may be filled with a matrix or substrate such as agar and a mixture of specific ingredients that may include nutrients, blood, salts, carbohydrates, dyes, indicators, amino acids and/or antibiotics. By inoculating the cavity with a microbe-laden sample, the growth and/or presence of microbes may be monitored.

Although the human eye is still widely used for microbe detection, there is a tendency towards automated systems as they can detect the microbial growth at a much earlier stage and preferably without human intervention. In such automated systems, a camera system may take pictures, preferably microscopic pictures, of the upper surface of the matrix or substrate at different moments in time. By comparing pictures of the same area and identifying visual differences microbial growth can be detected.

A drawback of the automated systems, which use a camera to make pictures of the upper surface of the matrix or substrate, is that the signal-to-noise ratio of the pictures is small.

### SUMMARY OF THE INVENTION

Hence, it is an object of the invention to increase the signal-to-noise ratio of automated systems used for visually monitoring organism growth in a container.

According to a first aspect of the invention, this object is achieved by a container for culturing organisms, comprising:
a holder defining a cavity; and
a separate lid to close off the cavity, said lid comprising a transparent window portion to be arranged above the cavity when the lid closes off the cavity, said window portion comprising an upper surface and a lower surface extending parallel to each other;
   wherein the cavity is filled with a matrix having a top surface on which an organism to be cultured can be received,
   characterized in that in at least one position of the lid with respect to the holder, in which the lid closes off the cavity, the top surface of the matrix extends non-parallel to the upper and lower surfaces of the window portion.

An advantage of the container according to the first aspect of the invention is that radiation incident to the window portion in a direction normal to the top surface of the matrix travels in a different direction after reflecting of the window portion than radiation passing the window portion in a direction normal to the top surface of the matrix. In this way radiation reflecting of the window portion, which radiation does not contain relevant information about the content of the container, is separated from radiation reflected of the top surface of the matrix and passing the window portion in a direction normal to the top surface of the matrix, which reflected radiation carries information about the presence of organism growth on the top surface of the matrix, so that a camera capturing the reflected radiation is subject to a higher signal-to-noise ratio compared to prior art containers in which the upper and lower surfaces of the window portion are parallel to the top surface of the matrix.

The container may have any shape in top view, including rectangular and circular shapes, but other shapes are also envisaged.

In an embodiment, the holder defines a supporting surface for supporting the holder, and wherein the top surface of the matrix extends parallel to the supporting surface. This has the advantage that the container does not have to be oriented properly with respect to a camera of a monitoring system to obtain a proper image. Once the camera is aligned with a support engaging with the supporting surface of the container, the camera is also automatically aligned with the top surface of the matrix of a container positioned on the support.

In an embodiment, the holder defines a supporting surface for supporting the holder, wherein the cavity is delimited by a bottom surface, and wherein the bottom surface extends parallel to the supporting surface.

In an embodiment, the holder defines a supporting surface for supporting the holder, and wherein in said at least one position the upper and lower surfaces of the window portion extend parallel to the supporting surface. This has the advantage that the container can easily be stacked.

The supporting surface defined by the holder, such that the holder can be supported from a support, e.g. a table, may be formed by a single support surface engaging with the support, but may also be formed by multiple separate support surfaces, e.g. due to the use of legs, feet, pins or the like, wherein the multiple support surfaces lie in the same plane. Hence, alternatively it can be said that the top surface of the matrix, the bottom surface of the cavity and/or the upper and lower surfaces of the window portion are parallel to the plane defined by the one or more support surfaces.

In an embodiment, the top surface of the matrix extends non-parallel to the upper and lower surfaces of the window portion in all angular positions of the lid with respect to the holder in which angular positions the lid closes off the cavity. This has the advantage that the container is always configured for optimal monitoring by a monitoring system.

In an embodiment, the container is configured such that in at least one other position of the lid with respect to the holder, in which the lid closes off the cavity of the holder, the upper and lower surfaces of the window portion are substantially parallel to the supporting surface defined by the holder for stacking purposes. This embodiment has the advantage that the container has two configurations, one in which the container is optimal for monitoring by a monitoring system and one in which the containers can be stacked.

In an embodiment, the lid and holder are identical. This makes the fabrication of the container easy, as only one type of component needs to be fabricated, wherein two of these components can form a container according to the first aspect of the invention.

In an embodiment, the holder defines multiple cavities, wherein the lid is configured to close off the multiple cavities. As a result, multiple cavities can be handled at the same time by handling the container, thereby increasing the speed of the monitoring system. In other words, the holder may define multiple cavities, preferably arranged in an array, as in microtiter plates. In case of multiple cavities, the cavities may be closed off by a single lid comprising a window portion for each cavity, or each cavity has its own separate lid.

It is explicitly noted here that closing off the cavity is not to be interpreted as not allowing any ventilation. Closing off the cavity is to be interpreted such that the content in the cavity is not easily accessible by a user.

In an embodiment, the holder comprises only one cavity as in a traditional petri dish.

In an embodiment, the container comprises glass and/or plastic. Besides the window portion being transparent, the entire lid may transparent. The holder may be transparent, but may also be opaque.

In an embodiment, the container is a shallow container, such that the thickness of the container is at most 0.5 times a width or a length of the container, preferably 0.3 times the width or length, and more preferably at most 0.2 times the width or length.

In an embodiment, the upper and lower surface of the window portion make an angle with the top surface of the matrix in the range of 10-200 mrad.

In an embodiment, the holder comprises a bottom portion arranged below the cavity, said bottom portion comprising a bottom surface delimiting the cavity and a lower surface opposite the bottom surface, wherein the bottom surface and the lower surface of the bottom portion extend parallel to each other. As a result thereof the bottom portion at the cavity has a constant thickness thereby reducing optical disturbances caused by the bottom portion.

In an embodiment, the bottom portion is free of text, drawings, symbols, prints and the like. This will keep the optical disturbances to a minimum.

In an embodiment, the bottom surface and the lower surface of the bottom portion extend non-parallel to the top surface of the matrix. In this way, any reflections of the bottom surface and/or lower surface of the bottom portion are separated from the reflection of interest of the top surface of the matrix.

In an embodiment, the bottom surface and the lower surface of the bottom portion extend parallel to the upper and lower surfaces of the window portion. In this way, the behaviour of reflections of the bottom surface and the lower surface of the bottom portion and the reflections of the upper and lower surfaces of the window portion is similar.

In an embodiment, the bottom portion comprises light absorbing material to reduce the optical disturbances caused by the bottom portion.

The second aspect of the invention also relates to a method for monitoring the culturing of organisms in a container, said method comprising the following steps:
a. providing a container according to a first aspect of the invention;
b. loading the top surface of the matrix with an organism-laden sample;
c. closing off the cavity using the lid of the container; and
d. making an image of the top surface of the matrix through the window portion by capturing light reflecting of the top surface and traveling in a direction normal to the top surface of the matrix.

In an embodiment, light is directed towards the top surface of the matrix through the window portion in a direction perpendicular to the top surface of the matrix to be reflected of the top surface and to be captured to make the image.

In an embodiment, the container is configured such that in a first position of the lid with respect to the holder, in which the lid closes off the cavity of the holder, the upper and lower surfaces of the window portion are substantially parallel to the top surface of the matrix, and such that in a second position of the lid with respect to the holder, in which the lid closes off the cavity of the holder, the upper and lower surfaces of the window portion are substantially non-parallel to the top surface of the matrix, and wherein the method comprises the step of providing the lid in the second position prior to step d.

In an embodiment, step d. is performed at different moments in time and the method further comprises the step of comparing images of the same area for visual differences indicating organism growth.

The first aspect of the invention further relates to a monitoring system for monitoring the culturing of organisms, said monitoring system comprising:
a container according to the first aspect of the invention;
a camera;
an objective to capture light reflected of the top surface of the matrix and to direct the captured light towards the camera; and
a light source to emit light towards the container to illuminate the top surface of the matrix,
   characterized in that an angle between the top surface of the matrix and the upper and lower surfaces of the window portion is larger than a numerical aperture of the objective.

In an embodiment, the objective comprises a telecentric lens.

According to a second aspect of the invention, the object is achieved by a container for culturing organisms, comprising:
a holder defining a supporting surface for supporting the holder and a cavity for receiving an organism to be cultured, wherein a bottom surface delimiting the cavity is parallel to the supporting surface; and
a separate lid to close off the cavity, said lid comprising a transparent window portion to be arranged above the cavity when the lid closes off the cavity, said window portion comprising an upper surface and a lower surface extending parallel to each other,
   characterized in that the container is configured such that in at least one position of the lid with respect to the holder, in which the lid closes off the cavity of the holder, the upper and lower surfaces of the window portion are substantially non-parallel to the bottom surface of the cavity.

An advantage of the container according to the second aspect of the invention is that radiation incident to the window portion in a direction normal to the bottom surface after reflecting of the window portion travels in a different direction than radiation passing the window portion in a direction normal to the bottom surface. In this way radiation reflecting of the window portion, which radiation does not contain relevant information about the content of the container, is separated from radiation passing the window portion in a direction normal to the bottom surface, which radiation carries information about the content of the container, so that a camera capturing the information carrying radiation is subject to a higher signal-to-noise ratio compared to prior art containers in which the upper and lower surfaces of the window portion are parallel to the bottom surface.

The container may have any shape including rectangular and circular shapes, but other shapes are also envisaged.

The holder may define a single cavity as in a traditional petri dish, but may also define multiple cavities, preferably arranged in an array, as in microtiter plates. In case of multiple cavities, the cavities may be closed off by a single lid comprising a window portion for each cavity, or each cavity has its own separate lid.

The supporting surface defined by the holder such that the holder can be supported from a support element, e.g. a table, may be formed by a single support surface engaging with the support element, but may also be formed by multiple separate support surfaces, e.g. due to the use of legs, feet, pins, and/or the like, wherein the multiple support surfaces lie in the same plane. Hence, alternatively it can be said that the bottom surface of the cavity defined by the holder is parallel to the plane defined by the one or more support surface.

In an embodiment, the container is a petri dish or a microtiter plate.

In an embodiment, the container comprises glass and/or plastic.

In an embodiment, the entire container is made of transparent material.

In an embodiment, the container is a shallow container, wherein a thickness of the container is at most 0.5 times a width or a length of the container, preferably at most 0.3 times the width or length, and more preferably at most 0.2 times the width or length.

In an embodiment, the holder defines an engagement surface for engaging with the lid when the lid closes off the cavity, wherein the engagement surface is parallel to the supporting surface.

In an embodiment, the holder defines an engagement surface for engaging with the lid when the lid closes off the cavity, wherein the engagement surface is non-parallel to the supporting surface.

In an embodiment, the engagement surface is parallel to the upper surface and the lower surface of the window portion.

In an embodiment, the engagement surface is non-parallel to the upper surface and the lower surface of the window portion.

In an embodiment, the container is configured such that in at least one other position of the lid with respect to the holder, in which the lid closes off the cavity of the holder, the upper and lower surfaces of the window portion are substantially parallel to the bottom surface of the cavity for stacking purposes.

In an embodiment, the lid and holder are identical.

In an embodiment, the cavity is filled with a matrix, wherein an upper surface of the matrix is parallel to the supporting surface.

In an embodiment, the upper and lower surface of the window portion make an angle with the bottom surface of the cavity in the range of 10-100 mrad, preferably 75 mrad.

In an embodiment, the lid comprises a first part and a separate second part, wherein when the lid closes off the cavity, the second part is arranged in between the first part and the holder and causes the upper and lower surfaces of the window portion to extend non-parallel to the bottom surface.

In an embodiment, the first part is able to close off the cavity without using the second part, in which case the upper and lower surfaces of the window portion are parallel to the bottom surface.

The second aspect of the invention also relates to a method for monitoring the culturing of organisms in a container, comprising the following steps:
a. providing a container according to the invention;
b. loading the cavity of the container with an organism-laden sample;
c. closing off the cavity using the lid of the container; and
d. imaging the organisms in the cavity through the window portion by capturing light coming from the container and traveling in a direction normal to the bottom surface of the cavity.

In an embodiment, the container is configured such that in a first position of the lid with respect to the holder, in which the lid closes off the cavity of the holder, the upper and lower surfaces of the window portion are substantially parallel to the bottom surface of the cavity, and such that in a second position of the lid with respect to the holder, in which the lid closes off the cavity of the holder, the upper and lower surfaces of the window portion are substantially non-parallel to the bottom surface of the cavity, and wherein the method comprises the step of providing the lid in the second position prior to step d.

In an embodiment, step d. is performed at different moments in time and the method further comprises the step of comparing images of the same area for visual differences indicating organism growth.

The second aspect of the invention further relates to a monitoring system for monitoring the culturing of organisms, said monitoring system comprising:
a container according to the first aspect of the invention;
a camera;
an objective to capture light from the container and to direct the captured light towards the camera; and
a light source to emit light towards the container to illuminate the container, characterized in that an angle between the bottom surface of the cavity and the upper and lower surfaces of the window portion is larger than a numerical aperture of the objective.

In an embodiment, the objective comprises a telecentric lens.

### BRIEF DESCRIPTION OF THE DRAWINGS

The invention according to the first and second aspect will now be described by reference to the accompanying drawings in which like parts are indicated by like reference symbols, and in which:

FIG. 1 depicts a prior art monitoring system to monitor the culturing of organisms;

FIG. 2 depicts a monitoring system to monitor the culturing of organisms using a container;

FIG. 3 depicts a container according to another embodiment;

FIG. 4 depicts a container according to a further embodiment;

FIG. 5 depicts a container according to an embodiment;

FIG. 6 depicts a container according to yet another embodiment; and

FIG. 7 depicts a container according to a further embodiment.

### DETAILED DESCRIPTION OF THE INVENTION

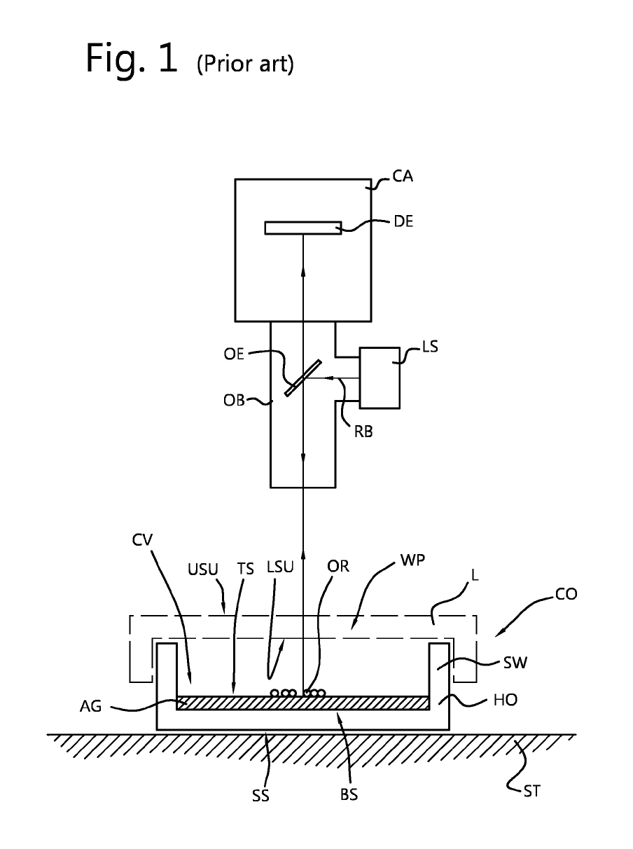

FIG. 1 schematically depicts a prior art monitoring system to monitor the culturing of organisms. The monitoring system comprises a camera CA, an objective OB and a light source LS.

The light source LS is configured to provide a radiation beam RB to the objective OB, which objective OB comprises an optical element OE to direct the radiation beam parallel to an optical axis of the objective. Hence, light is emitted from the objective. The objective is configured to capture an image of reflected light, which light passes the optical element OE and is incident to a detector DE of the camera CA.

The prior art monitoring system uses prior art containers CO for culturing organisms, wherein the container CO comprises a holder HO and a lid L. The holder HO defines a supporting surface SS for supporting the holder HO, in this embodiment, of a support ST. The holder HO further defines a cavity CV for receiving an organism OR to be cultured, said cavity being delimited at one side by a bottom surface BS that is parallel to the supporting surface SS. The cavity CV is further delimited by a sidewall SW.

In this embodiment, the cavity is filled with a matrix, in this case a layer of agar AG, preferably comprising specific ingredients such as nutrients, blood, salts, carbohydrates, dyes, indicators, amino acids and/or antibiotics. Agar is usually provided to the container in warm liquid form. Once the agar solidifies it can be inoculated with an organism-laden sample. The inoculated organism may be the organism of interest, but the organism may also be provided as a host for a virus or phage, so that a second inoculation is required to introduce the virus or phage of interest. The agar will have a top surface TS that is substantially parallel to the supporting surface SS and the bottom surface BS.

The lid L is separate from the holder HO in that it is not connected to the holder HO via hinges or any other structure. The lid L can thus be separated from the holder HO without damaging the container. The lid is configured to close off the cavity so that the organism can be cultured without the risk of contamination. The content of the cavity may for instance not be contaminated by airborne particles and organisms in the environment of the container, but alternatively or additionally the environment may not be contaminated by the organisms inside the cavity of the container.

The lid comprises a transparent window portion WP to be arranged above the cavity CV when the lid L closes off the cavity CV, wherein said window portion WP comprises an upper surface USU and a lower surface LSU extending parallel to each other and parallel to the top surface TS, the supporting surface SS and the bottom surface BS.

When the container CO is positioned beneath the monitoring system of FIG. 1, the radiation beam RB directed towards the container is preferably directed perpendicular to the top surface TS of the agar, such that the reflection of the radiation beam at the top surface is also perpendicular and can be captured by the objective OB. However, when the lid is present as shown in FIG. 1, a portion of the radiation beam may reflect of the upper surface USU of the lid and a portion of the radiation beam may reflect of the lower surface LSU of the lid. These reflections will also be captured by the objective OB, but do not carry any information about the presence or absence of the organism inside the container CO, so that the signal-to-noise ratio is relatively low.

It may be obvious to improve the signal-to-noise ratio by removing the cause of the reflections. That is, the lid is removed of the holder during monitoring by the monitoring system. This is why the lid is drawn in dashed lines in FIG. 1. Removing the lid may solve the problem of the reflections, but the risk of contamination is then highly increased. This solution is thus not preferred.

Another, possibly obvious, solution may be provided by looking at the solutions provided by users of microscopes which also try to look at the organism through the lid. From this field it is known to tilt the entire container. This may cause the reflections to be directed away from the objective, so that they cannot be captured by the objective, but makes positioning and orienting the matrix relative to the camera and objective more challenging. Hence, these solutions are also not preferred.

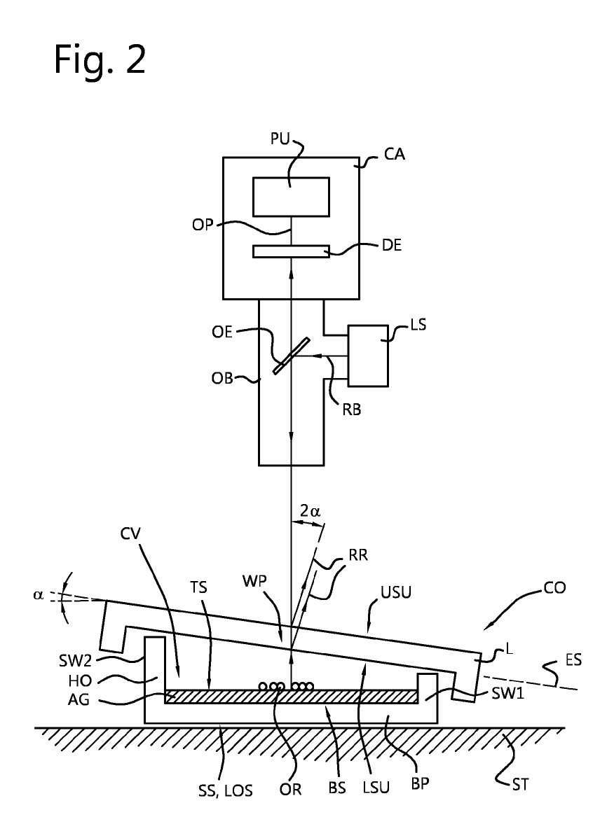

The inventors have come up with the idea to adjust the container, such that in at least one position of the lid with respect to the holder, in which the lid closes off the cavity of the holder, the upper and lower surfaces of the window portion are substantially non-perpendicular to the light emitted by the camera, i.e. are non-perpendicular to an optical axis defined by the objective. This will be explained in more detail with respect to FIG. 2 and further.

FIG. 2 depicts a monitoring system according to an embodiment of the invention, which monitoring system is suitable to carry out a method according to the invention using a container CO according to the invention.

As in FIG. 1, the monitoring system comprises a camera CA, an objective OB and a light source LS.

The light source LS is configured to provide a radiation beam RB to the objective OB, which objective OB comprises an optical element OE to direct the radiation beam parallel to an optical axis of the objective. Hence, light is emitted from the objective. The objective is configured to capture an image of reflected light, which light passes at least partially the optical element OE and is incident to a detector DE of the camera CA. An output OP of the detector DE is processed in a processing unit PU.

The monitoring system is used in combination with a container CO according to an embodiment of the invention for culturing organisms, wherein the container CO comprises a holder HO and a lid L. The holder HO defines a supporting surface SS for supporting the holder HO, in this embodiment, from a support ST. The holder HO further defines a cavity CV for receiving an organism OR to be cultured, said cavity being delimited at one side by a bottom surface BS that in this embodiment is parallel to the supporting surface SS. The cavity CV is further delimited by a sidewall extending from the bottom surface BS, which sidewall comprises a first sidewall portion SW1 and an second sidewall portion SW2 opposite to the first sidewall portion SW1.

In this embodiment, the cavity is filled with matrix, e.g. a layer of agar AG preferably comprising specific ingredients such as nutrients, blood, salts, carbohydrates, dyes, indicators, amino acids and/or antibiotics. Agar is usually provided to the container in a warm liquid form. Once the agar solidifies it can be inoculated with an organism-laden sample. The inoculated organism may be the organism of interest, but the organism may also be provided as a host for a virus or phage, so that a second inoculation is required to introduce the virus or phage of interest. The agar AG has a top surface TS that is in this embodiment substantially parallel to the supporting surface SS an the bottom surface BS.

The lid L is separate from the holder HO in that it is not connected to the holder HO via hinges or any other structure. The lid L can thus be separated from the holder HO without damaging the container. The lid is configured to close off the cavity so that the organism can be cultured without the risk of contamination.

The lid comprises a transparent window portion WP to be arranged above the cavity CV when the lid L closes off the cavity CV, wherein said window portion WP comprises an upper surface USU and a lower surface LSU extending parallel to each other.

The container CO is configured such that in at least one position of the lid L with respect to the holder HO, in which the lid L closes off the cavity CV of the holder HO, the upper and lower surfaces USU, LSU of the window portion WP are substantially non-parallel to the bottom surface BS of the cavity CV, i.e. the upper and lower surfaces USU, LSU of the window portion make a non-zero angle α with the bottom surface BS. Hence, FIG. 2 discloses a container according to the second aspect of the invention. However, as the top surface TS of the agar is also parallel to the bottom surface BS, the upper and lower surfaces USU, LSU of the window portion also make a non-zero angle α with the top surface TS of the matrix. Hence, FIG. 1 also discloses a container according to the first aspect of the invention.

In the embodiment of FIG. 2, the tilted orientation of the lid is caused by the non-equal heights of the first and second side wall portions SW1, SW2. The height of the second side wall portion SW2 is larger than the corresponding height of the first side wall portion SW1. The height of the sidewall of the holder HO may gradually change from the indicated height of the second side wall portion SW2 to the first side wall portion SW1.

When the container CO is positioned beneath the monitoring system of FIG. 2, the radiation beam RB directed towards the container CO is directed perpendicular to the top surface TS of the agar, i.e. perpendicular to the bottom surface BS of the container CO, such that the reflection of the radiation beam of the top surface TS is also perpendicular and can be captured by the objective OB.

Radiation is also partially reflected of the upper surface USU and the lower surface LSU of the window portion WP. However, due to the tilt of the lid with respect to the horizontal the reflected radiation RR is reflected away from the vertical at an angle substantially equal to 2*α, which prevents the objective OB from capturing the reflected radiation RR when the numerical aperture of the objective is smaller than angle α, which increases the signal-to-noise ratio.

The embodiment of FIG. 2 is such that the lid L is tilted in any position of the lid L relative to the holder HO in which the lid L closes off the cavity CV.

In an embodiment, the objective OB may be telecentric.

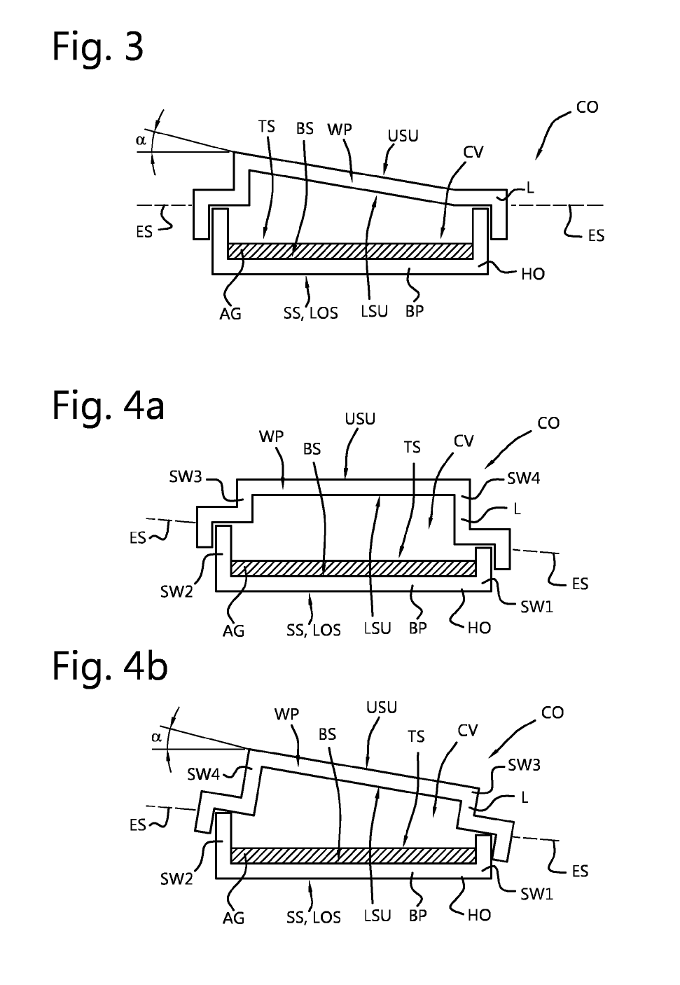

FIG. 3 depicts a container CO according to another embodiment of the invention. The container comprises a holder HO defining a supporting surface SS for supporting the holder HO and a cavity CV for receiving an organism (not shown) to be cultured, wherein a bottom surface BS delimiting the cavity is parallel to the supporting surface SS.

The container CO further comprises a separate lid L to close off the cavity CV, said lid L comprising a transparent window portion WP to be arranged above the cavity CV when the lid L closes off the cavity CV, as shown in FIG. 3, said window portion WP comprising an upper surface USU and a lower surface LSU extending parallel to each other.

The container CO is configured such that in at least one position of the lid L with respect to the holder HO, in which the lid L closes off the cavity CV of the holder HO, the upper and lower surfaces USU, LSU of the window portion WP are substantially non-parallel to the bottom surface BS of the cavity CV, i.e. the upper and lower surfaces USU, LSU of the window portion WP make a non-zero angle α with the bottom surface BS. Hence, FIG. 3 discloses a container according to the second aspect of the invention.

The cavity CV is filled with a matrix or substrate, in this embodiment agar AG with additives enabling the growth of organisms on the agar AG. The agar is a layer of material having a top surface TS extending in this embodiment parallel to the bottom surface BS. Hence, FIG. 3 also discloses a container according to the first aspect of the invention.

In the embodiment of FIG. 3, the tilted orientation of the window portion WP is implemented in the lid L. In other words, the holder HO in the embodiment of FIG. 3 defines an engagement surface indicated by plane ES for engaging with the lid L when the lid L closes off the cavity CV, wherein the engagement surface is parallel to the supporting surface SS, but the upper surface USU and the lower surface LSU are non-parallel to the engagement surface.

In the embodiment of FIG. 2, in which the tilted orientation of the window portion is implemented in the holder HO, the holder HO defines an engagement surface indicated by plane ES for engaging with the lid L when the lid L closes off the cavity CV, wherein the engagement surface is non-parallel to the supporting surface SS, but the upper surface USU and the lower surface LSU are parallel to the engagement surface.

In the embodiments of both FIGS. 2 and 3, the window portion WP has a tilted orientation independent of the angular position of the lid with respect to the holder about an axis perpendicular to the bottom surface BS. This is in these embodiments, in which the top surface of the agar is parallel to the bottom surface, caused by either the engagement surface being parallel to the bottom surface (embodiment of FIG. 3) or the engagement surface being parallel to the upper and lower surface USU, LSU (embodiment of FIG. 2).

FIG. 4 depicts a depicts a container CO according to a further embodiment of the invention in two configurations, namely a configuration A and a configuration B. The container comprises a holder HO defining a supporting surface SS for supporting the holder HO and a cavity CV for receiving an organism (not shown) to be cultured, wherein a bottom surface BS delimiting the cavity is parallel to the supporting surface SS.

The container CO further comprises a separate lid L to close off the cavity CV, said lid L comprising a transparent window portion WP to be arranged above the cavity CV when the lid L closes off the cavity CV, as shown in both configurations of FIG. 4, said window portion WP comprising an upper surface USU and a lower surface LSU extending parallel to each other.

The holder HO comprises a sidewall including a first sidewall portion SW1 and a second sidewall portion SW2, wherein the height of the second sidewall portion is larger than the height of the first sidewall portion.

The lid comprises a sidewall including a third sidewall portion SW3 and a fourth sidewall portion SW4, wherein the height of the fourth sidewall portion SW4 is larger than the height of the third sidewall portion SW3.

The container is configured such that in a first position of the lid L with respect to the holder HO corresponding to configuration A in which the lid closes off the cavity, the upper surface USU and the lower surface LSU are parallel to the bottom surface. This configuration is advantageous as it allows stacking of multiple containers CO when the containers are all in configuration A. Configuration A may be obtained by choosing the sum of the heights of the first and fourth sidewall portions SW1, SW4 to be equal to the sum of the heights of the second and third sidewall portions SW2, SW3.

Configuration B depicts a second position of the lid L with respect to the holder HO. In configuration B, the lid is rotated 180 degrees with respect to configuration A about an axis perpendicular to the bottom surface BS. Hence, the fourth sidewall portion SW4 is situated above the second sidewall portion SW2 and the third sidewall portion SW3 is situated above the first sidewall portion SW1. As the fourth and second sidewall portions SW4, SW2 are larger than the corresponding third and first sidewall portions SW3, SW1, the upper and lower surfaces USU, LSU of the window portion WP are substantially non-parallel to the bottom surface BS of the cavity CV, i.e. the upper and lower surfaces USU, LSU of the window portion WP make a non-zero angle α with the bottom surface BS, in the second position of the lid with respect to the holder. Hence, FIG. 4 discloses a container according to the second aspect of the invention.

The cavity CV is filled with a matrix or substrate, in this embodiment agar AG with additives enabling the growth of organisms on the agar AG. The agar is a layer of material having a top surface TS extending in this embodiment parallel to the bottom surface BS. Hence, FIG. 4 also discloses a container according to the first aspect of the invention.

In the embodiment of FIG. 4, the tilted orientation of the window portion is dependent on the orientation of the lid with respect to the holder. This is due to the fact that the holder defines an engagement surface indicated by plane ES that is non-parallel to the bottom surface BS and top surface TS, but is also non-parallel to the upper surface USU and the lower surface LSU.

In an embodiment closely related to the embodiment shown in FIG. 4, the lid and holder are identical, which has the advantage that only a single component needs to be fabricated over and over again to mass produce the containers. In case the lid and holder are fabricated using mould injection, only one mould is required to fabricate both the lid and holder.

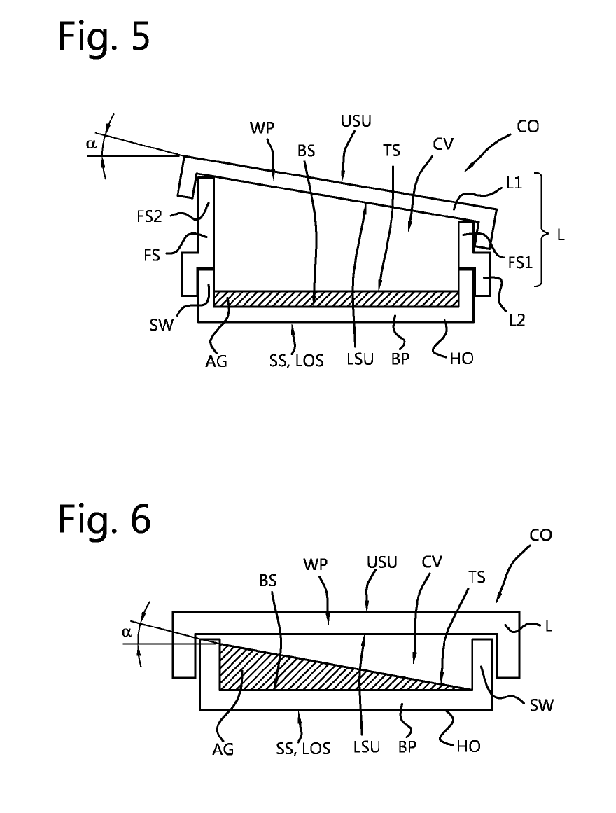

FIG. 5 depicts a container CO according to an embodiment of the invention. The container comprises a holder HO defining a supporting surface SS for supporting the holder HO and a cavity CV for receiving an organism (not shown) to be cultured, wherein a bottom surface BS delimiting the cavity is parallel to the supporting surface SS.

The container CO further comprises a separate lid L to close off the cavity CV. In this embodiment, the lid L comprises a first part L1 and a second part L2. When the lid closes off the cavity, the second part L2 is arranged in between the holder HO and the first part L1. The first and second part L1, L2 may be separate components that are to be assembled when the cavity CV needs to be closed.

An advantage of using a lid L with two parts L1, L2, is that the first part L1 and the holder may be formed by a prior art container, e.g. a petri dish, which is adapted by providing and using the second part L2 dedicated for implementing the invention.

The first part L1 of the lid L comprises a transparent window portion WP to be arranged above the cavity CV when the lid L closes off the cavity CV, as shown in FIG. 5, said window portion WP comprising an upper surface USU and a lower surface LSU extending parallel to each other.

The container CO is configured such that in at least one position of the lid L with respect to the holder HO, in which the lid L closes off the cavity CV of the holder HO, the upper and lower surfaces USU, LSU of the window portion WP are substantially non-parallel to the bottom surface BS of the cavity CV, i.e. the upper and lower surfaces USU, LSU of the window portion WP make a non-zero angle α with the bottom surface BS. Hence, FIG. 5 discloses a container according to the second aspect of the invention.

The cavity CV is filled with a matrix or substrate, in this embodiment agar AG with additives enabling the growth of organisms on the agar AG. The agar is a layer of material having a top surface TS extending in this embodiment parallel to the bottom surface BS. Hence, FIG. 5 also discloses a container according to the first aspect of the invention.

In the embodiment of FIG. 5, the tilted orientation of the window portion WP is implemented in the lid L, more in particular in the second part L2. The second part L2 extends a sidewall SW of the holder HO by providing a further sidewall FS with a first sidewall portion FS1 and a second sidewall portion FS2 opposite the first sidewall portion FS1. The height of the second sidewall portion FS2 is larger than the height of the first sidewall portion FS1 thereby introducing the tilt in the first part L1 which is supported by the second part L2.

In the embodiments of FIG. 5, the window portion WP has a tilted orientation independent of the angular position of the lid with respect to the holder about an axis perpendicular to the bottom surface BS.

FIG. 6 depicts a CO according to an embodiment of the invention. The container comprises a holder HO defining a supporting surface SS for supporting the holder HO and a cavity CV for receiving an organism (not shown) to be cultured, wherein a bottom surface BS delimiting the cavity is parallel to the supporting surface SS.

The container CO further comprises a separate lid L to close off the cavity CV, said lid L comprising a transparent window portion WP to be arranged above the cavity CV when the lid L closes off the cavity CV, as shown in FIG. 6, said window portion WP comprising an upper surface USU and a lower surface LSU extending parallel to each other and extending parallel to the bottom surface BS. Hence, the holder and lid of container CO form a traditional container.

The cavity CV is filled with a matrix or substrate, in this embodiment agar AG with additives enabling the growth of organisms on the agar AG. The agar is a layer of material having a top surface TS extending in this embodiment non-parallel to the upper and lower surfaces USU, LSU of the window portion WP. Hence, FIG. 6 depicts a container according to the first aspect of the invention and not according to the second aspect of the invention.

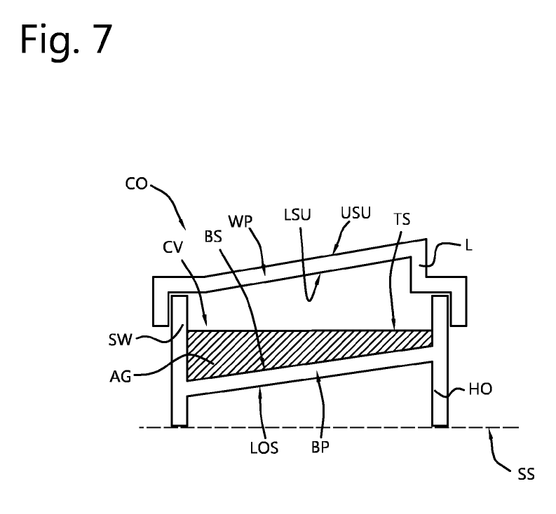

FIG. 7 depicts a CO according to an embodiment of the invention. The container comprises a holder HO defining a supporting surface SS for supporting the holder HO and a cavity CV for receiving an organism (not shown) to be cultured. The cavity CV is delimited by a bottom portion BP arranged below the cavity CV and a sidewall SW, wherein the bottom portion comprises a bottom surface BS and a lower surface LOS opposite the bottom surface, wherein the bottom surface and lower surface LOS extend parallel to each other, but non-parallel to the supporting surface SS. In the embodiments of FIG. 2-6, the supporting surface SS was formed by the lower surface LOS.

The container CO further comprises a separate lid L to close off the cavity CV, said lid L comprising a transparent window portion WP to be arranged above the cavity CV when the lid L closes off the cavity CV, as shown in FIG. 7, said window portion WP comprising an upper surface USU and a lower surface LSU extending parallel to each other.

The cavity CV is filled with a matrix or substrate, in this embodiment agar AG with additives enabling the growth of organisms on the agar AG. The agar is a layer of material having a top surface TS extending in this embodiment non-parallel to the upper and lower surfaces USU, LSU of the window portion WP. Hence, FIG. 6 depicts a container according to the first aspect of the invention.

An advantage of the tilted window portion of the lid and the tilted bottom portion of the holder is that when radiation incident to these parts in a direction normal to the top surface of the agar is reflected, the radiation is reflected in a different direction than radiation reflecting of the top surface of the agar and passing the window portion towards a camera of a monitoring system.

Although the description and examples are about arranging organisms on a top surface of a matrix or substrate, the invention is not limited to only arranging the organisms on this top surface. It is thus also possible that a filter is provided on top of the matrix or substrate, wherein the organisms are positioned on top of the filter, and wherein the filter is permeable for components of the matrix or substrate, so that the organisms can use these components for culturing.

1. A container for culturing organisms, comprising:
a holder defining a cavity; and
a separate lid to close off the cavity, said lid comprising a transparent window portion to be arranged above the cavity when the lid closes off the cavity, said window portion comprising an upper surface and a lower surface extending parallel to each other;
wherein the cavity is filled with a matrix having a top surface on which an organism to be cultured can be received, and
wherein in at least one position of the lid with respect to the holder, in which the lid closes off the cavity, the top surface of the matrix extends non-parallel to the upper and lower surfaces of the window portion.

2. The container according to claim 1, wherein the holder defines a supporting surface for supporting the holder, and wherein the top surface of the matrix extends parallel to the supporting surface.

3. The container according to claim 1, wherein the holder defines a supporting surface for supporting the holder, wherein the cavity is delimited by a bottom surface, and wherein the bottom surface extends parallel to the supporting surface.

4. The container according to claim 1, wherein the holder defines a supporting surface for supporting the holder, and wherein in said at least one position the upper and lower surfaces of the window portion extend parallel to the supporting surface.

5. The container according to claim 1, wherein the top surface of the matrix extends non-parallel to the upper and lower surfaces of the window portion in all angular positions of the lid with respect to the holder in which angular positions the lid closes off the cavity.

6. The container according to claim 2, wherein the container is configured such that in at least one other position of the lid with respect to the holder, in which the lid closes off the cavity of the holder, the upper and lower surfaces of the window portion are substantially parallel to the supporting surface defined by the holder for stacking purposes.

7. The container according to claim 1, wherein the lid and holder are identical.

8. The container according to claim 1, wherein the holder defines multiple cavities, and wherein the lid is configured to close off the multiple cavities.

9. The container according to claim 1, wherein the upper and lower surface of the window portion make an angle with the top surface of the matrix in the range of 10-200 mrad.

10. The container according to claim 1, wherein the holder comprises a bottom portion arranged below the cavity, said bottom portion comprising a bottom surface delimiting the cavity and a lower surface opposite the bottom surface, wherein the bottom surface and the lower surface of the bottom portion extend parallel to each other.

11. The container according to claim 10, wherein the bottom surface and the lower surface of the bottom portion extend non-parallel to the top surface of the matrix.

12. The container according to claim 11, wherein the bottom surface and the lower surface of the bottom portion extend parallel to the upper and lower surfaces of the window portion.

13. The container according to claim 10, wherein the bottom portion comprises light absorbing material.

14. A method for monitoring the culturing of organisms in a container, comprising the following steps:
providing a container according to claim 1;
loading the top surface of the matrix with an organism-laden sample;
closing off the cavity using the lid of the container; and
making an image of the top surface of the matrix through the window portion by capturing light reflecting of the top surface and traveling in a direction normal to the top surface of the matrix.

15. The method according to claim 14, wherein the container is configured such that in a first position of the lid with respect to the holder, in which the lid closes off the cavity of the holder, the upper and lower surfaces of the window portion are substantially parallel to the top surface of the matrix, and such that in a second position of the lid with respect to the holder, in which the lid closes off the cavity of the holder, the upper and lower surfaces of the window portion are substantially non-parallel to the top surface of the matrix, and wherein the method comprises the step of providing the lid in the second position prior to step d.

16. The method according to claim 14, wherein step d. is performed at different moments in time and the method further comprises the step of comparing images of the same area for visual differences indicating organism growth.

17. A monitoring system for monitoring the culturing of organisms, said monitoring system comprising:
a container according to claim 1;
a camera;
an objective to capture light reflected of the top surface of the matrix and to direct the captured light towards the camera; and
a light source to emit light towards the container to illuminate the top surface of the matrix,
wherein an angle between the top surface of the matrix and the upper and lower surfaces of the window portion is larger than a numerical aperture of the objective.

18. The monitoring system according to claim 17, wherein the objective comprises a telecentric lens.
